(19) United States
(12) Patent Application Publication
(10) Pub. No.: US 2026/0232905 A1
(43) Pub. Date: Aug. 13, 2026

(54) MEDICAMENT DELIVERY DEVICE AND MEDICAMENT DELIVERY ASSEMBLY

(71) Applicant: SHL Medical AG, Zug (CH)

(72) Inventor: Wen-An Chang, Taoyuan (TW)

(21) Appl. No.: 19/153,785

(22) PCT Filed: Jan. 26, 2024
(86) PCT No.: PCT/EP2024/051840

§ 371 Date: Aug. 5, 2025

(30) Foreign Application Priority Data
Feb. 9, 2023 (EP) 23155785.1

(51) Int. Cl. A61M 5/20; A61M 5/28; A61M 5/315; A61M 5/32

(52) U.S. Cl. CPC A61M 5/2033; A61M 5/288; A61M 5/31501; A61M 5/31571; A61M 5/31581; A61M 5/3202; A61M 2005/2006; A61M 2005/2073; A61M 2202/0007

(57) ABSTRACT

A medicament delivery device according to the present text is equipped to accommodate a medicament container so as to form, together with the medicament container, a medicament delivery assembly. The medicament delivery device comprises a housing that has a proximal end and a distal end and defines an axis that extends proximodistally. It further comprises an expelling unit for expelling a medicament from a medicament container seated within the housing, the expelling unit comprising plunger element equipped to be driven in an axial direction while acting on the medicament container to expel the medicament. The medicament delivery device further comprises an activator The activator is equipped to prevent the expelling unit from expelling the medicament unless it is rotated about the axis by a certain angle. An activation sub-assembly, which may comprise a needle guard link connected to a needle guard, causes the rotation of the activator about the axis when subject to an axial application movement caused by the user.

### TECHNICAL FIELD

The invention is in the field of medicament delivery devices. More in particular, it relates to an automatic medicament delivery device being safe and easy to handle.

### BACKGROUND

Among the medicament delivery devices for self-administration, there are medicament delivery devices that have a needle pre-attached thereto, for perforating, immediately prior to use, a septum of the medicament container accommodated in the medicament delivery device, and for delivering the medicament in the medicament container to a dose delivery site. Such medicament delivery devices may comprise a cap assembly with a needle cap that protects the needle before use. For use, the needle cap has to be removed.

WO 2013/089616 discloses an example of a medicament delivery device of this kind. The cap assembly comprises a needle hub, whereby the needle acts to pierce the septum upon removal of the needle cap. As a safety feature, the cap assembly comprises an outer cap in addition to the needle cap, with a cap clutch between the outer cap and the needle cap. For rotationally coupling the outer cap to the needle cap, a dose knob arranged at the distal of the medicament delivery device end has to be turned for unlocking. This turning releases a needle guard and allows it to travel in an axial direction to move a locking member of the clutch so as to rotationally lock the outer cap to the needle cap. Only then, by turning the outer cap, may the outer cap and the needle cap be removed. Also, by turning the outer cap together with the needle cap, the needle is caused to pierce the septum. The cap clutch is biased to prevent the locking member from getting, prior to the turning of the dose knob and without the user's intention, into the position in which it locks the inner and outer caps with respect to each other.

This solution is safe and reliable. However, there is still room for improvement in terms of ease of handling and in terms of complication, especially in view of the many sophisticated parts needed for the prior art medicament delivery devices.

### SUMMARY

Accordingly, it is an object of the present invention to provide a medicament delivery device that overcomes at least some of the drawbacks of the prior art. Especially, the medicament delivery device should be advantageous in terms of reliability, ease of handling and/or complexity.

This object is achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

According to an aspect of the present invention, a medicament delivery device is provided. The medicament delivery device is equipped to accommodate a medicament container so as to form, together with the medicament container, a medicament delivery assembly. The medicament delivery device comprises a housing that has a proximal end and a distal end and defines an axis that extends proximodistally. It further comprises an expelling unit for expelling a medicament from a medicament container seated within the housing, the expelling unit comprising plunger element equipped to be driven in an axial direction while acting on the medicament container to expel the medicament. The medicament delivery device further comprises an activator. The activator is equipped to prevent the expelling unit from expelling the medicament unless it is rotated about the axis by a certain angle. An activation sub-assembly causes the rotation of the activator about the axis when subject to an axial application movement caused by the user.

In the present text, the term ‘application movement’ is used to designate an axial movement of the activation sub-assembly, which movement causes the rotation of the activator.

Thus, in contrast to the prior art, no distal knob is required that has to be turned by the user. Rather, the user may cause the rotation of the activator by causing the axial movement. This may especially be a movement towards distally, so that the user simply has to push back the activation sub-assembly.

Thus, the medicament delivery device may be configured such that no active rotation of a distal knob or similar is required, and especially no active rotation by the user except a removal, for example unscrewing, movement of a proximal needle cap may be needed.

For causing the rotation, one of the activator and of the activation sub-assembly may comprise a bevel and the other one may comprise a bevel engaging structure facing towards the bevel. The application movement may cause the bevel and the bevel engaging structure to be pressed against each other, while the axial position of the activator relative to the housing is fixed, whereby a torque on the activator relative to the activation sub-assembly is generated and the bevel engaging structure slides along the bevel, so that the activator is rotated. The bevel may comprise a ramp running axially and circumferentially.

The bevel engaging structure may be a transmission protrusion being a radial protrusion. For example, the activator (or the activation sub-assembly) may comprise a cylindrical section, with the transmission protrusion protruding from the outer surface towards radially outwardly, when the activation sub-assembly (or the activator, respectively), has a cylindrical body portion, with the bevel that extends around the cylindrical section. Conversely, the transmission protrusion may protrude from an inner surface towards radially-inwardly if the cylindrical section carrying it extends around the cylindrical body portion.

The medicament delivery device may further comprise a needle protected by a needle cap. The needle may be held by a needle hub and may be caused to pierce a septum of the medicament container when the cap is removed. More in particular, the needle hub may be subject to an axial movement towards distally to pierce the septum when the cap is removed by an removal rotation, for example an unscrewing movement or a rotation to de-engage a bayonet coupling.

The medicament delivery device may further comprise an outer cap around the needle cap, coaxial with the needle cap. The needle cap has to be removed, together with the outer cap, before medicament delivery. To this end, the needle cap has to be rotated, for example, for unscrewing it. Because only the outer cap is directly accessible for the user, for removing the needle cap together with the outer cap, the needle cap has to be rotationally coupled to the outer cap. In embodiments, such rotational coupling is only possible upon completion of the activation movement.

For example, in embodiments, the rotational coupling requires pushing the outer cap relative to the needle cap far enough and/or strong enough for also the activation sub-assembly to be pushed and to be subject to the application movement so that the needle cap can only be removed if the activation sub-assembly has cooperated with the activator to rotate the activator.

For example, in a preferred example, the needle cap comprises ratchet teeth circumferentially extending around an outer surface of a body of the needle cap; and the outer cap comprises counter ratchet teeth circumferential extending an inner surface of a body of the outer cap. In one example where the activation sub-assembly comprises a needle guard. The distal end of the outer cap is adjacent to the proximal end of the needle guard. When the user has not yet pressed the needle guard in the distal direction, the ratchet teeth of the needle cap is spaced apart from the counter ratchet teeth of the outer cap. As the outer cap is adjacent to the needle guard, the user can press the needle guard in the distal direction by pressing the outer cap. In this example, the application movement is the distal movement of the needle guard (for example, the needle guard comprises the bevel or the needle guard is operably connected to the bevel). Once the user pressed the needle guard in the distal direction, namely, performed the activation movement, the counter ratchet teeth of the outer cap is moved to line up with the ratchet teeth of the inner cap. As a result, the outer cap and the inner cap are rotationally coupled and can be rotated to remove.

In addition, or as an alternative, in embodiments, the rotational coupling between the outer cap and the needle cap directly requires the rotation of the activator, for example by a mechanism substantially as described in WO2013/089616.In this and other examples, the rotational coupling may be caused by a cap clutch. The cap clutch may be brought into an engaging position by pushing the outer cap relative to the needle cap, and/or by the rotation of the activator, for example by the activation sub-assembly being allowed to be moved, by a spring force, to a position in which it causes the clutch to be brought into the engaging position.

Thereby, it is ensured that the medicament container can only be pierced after the activator has been rotated by the angle. Before the medicament container (in particular a septum thereof) is pierced, the medicament cannot be expelled, even if the plunger element acts on the medicament container.

Ensuring that piercing the medicament container is only possible upon completion of the activation movement is a first way to ensure that rotation of the activator about the axis by an angle is a pre-condition for the unit to expel the medicament.

In addition, or as an alternative to this first way, the activator may, according to a second way, also directly interact with the expelling unit in that the activator limits movements of the plunger element into the axial, especially proximal, direction prior to the rotation of the activator. To this end, the activator may comprise a control structure, for example a ledge, especially an inward ledge if the plunger element runs through an interior of the activator. Such control structure may interact with a corresponding structure of the plunger element, for example a control protrusion (an outward protrusion if the plunger element runs through an interior of the activator, to prevent the plunger element from moving axially in the initial position of the activator. By the rotation of the activator, the control structure is brought out of engagement with the corresponding structure, and thereby the control structure does not prevent the plunger element from moving axially any more.

As an alternative to a dedicated control protrusion of the plunger element, also other structures may cooperate with the control structure to prevent the movement of the plunger element prior to the rotation of the activator—for example a control structure of the activator could be positioned between the drive spring and the plunger rod initially, or a control structure of the activator—being an inward protrusion-could initially be positioned between the medicament container and a proximal end of the plunger element.

According to the second way, in embodiments, after the rotation of the activator, a certain axial movement of the plunger element may be possible. Such axial movement of the plunger element may for example be limited by a control abutment.

Such control abutment may in embodiments be a feature of a separate element, for example of a plunger locking member, which may be rotatable about the axis, and which in the later expelling process may control the movement of the plunger element, activated for example by a pressing of the medicament delivery device against a dose delivery site and a displacement of a needle guard caused thereby.

In a group of alternative embodiments, a control abutment can be a feature of the activator itself. Then, in a sub-group of these alternative embodiments, the activator may be subject to a further rotation upon activation of the medicament delivery process after the priming process and when the medicament delivery device is ready for medicament delivery, for example by pushing the needle guard towards distally after removal of the needle cap. In this group of alternative embodiments, the functions of the activator and of a plunger locking member—sometimes called ‘rotator’ in devices of the kind described herein—may thus be combined in a single element—the activator may also function as rotator.

In embodiments, both, the first and the second way are implemented. For priming, the plunger element then presses against a stopper of the medicament container as soon as the activator has been subject to the rotation movement and the plunger element is released. When subsequently the needle is caused to penetrate the septum, the driving force onto the plunger element causes a certain movement of the stopper until the plunger element is stopped by a control abutment, whereby, when the medicament delivery assembly is held in the right position, with the distal end pointing upwardly, remaining air in the container and in the needle is expelled, for example together with a few droplets of the medicament.

In addition or as an alternative to priming, the rotation of the activator and a release of the plunger element caused thereby may cause a mixing of multiple substances and/or other step that has to be carried out prior to medicament delivery.

The activation sub-assembly may be arranged to be pushed into the axial, distal, direction by the user pushing the outer cap back towards distally.

Therefore, in embodiments the user just has to grip the outer cap, push it towards distally far enough to also push the activation sub-assembly so as to rotate the activator, and then to turn the outer cap until the outer cap together with the needle cap can be removed. This simple step of sequences, all carried out on the outer cap, and possibly all without the user unclasping the outer cap, all operations to be carried out for the medicament delivery assembly to be ready for injection.

In embodiments, the medicament delivery device comprises a needle guard that is configured to protect the needle after removal of the needle cap. The needle guard is configured to move into the proximal direction, for example activated by a needle guard spring, into an extended position in which it surrounds the proximal tip of the needle.

The activation sub-assembly may comprise the needle guard. I.e., what the user does when causing the application movement is, in these embodiments, pushing back towards distally the needle guard, for example by pushing the outer cap.

In embodiments, in addition to the activation sub-assembly, a pushing action causing the application movement may further cause a displacement of the medicament container relative to the container housing that holds it. Such movement of the medicament container may especially ensure that the plunger element—especially plunger rod—of the expelling unit gets into contact—or almost into contact—with a stopper of the medicament container as a step in preparing a priming operation. This may, in embodiments, be advantageous compared to solutions in which the plunger element is allowed to snap onto the plunger activated by a drive spring.

For example, the medicament delivery device may comprise a retainer into which the proximal end of the medicament container (for example its neck if the medicament container has a bottle-like shape) is introduced and relative to which an axial movement of the needle is caused for piercing the medicament container. Then, the retainer may be movable relative to the container housing. In embodiments, an engagement between the container housing and the retainer limits the movement of the medicament container relative to the housing and thereby may also limits the application movement.

In a group of alternative embodiments, the activation sub-assembly comprises for example a button arranged at the distal end of the housing, and the application movement may be a proximal movement relative to the activator, caused by the user pushing the button towards proximally. Thus, in these alternative embodiments, the activation sub-assembly does not require any needle guard—independent of whether such needle guard is present in the medicament delivery device or not.

The plunger element may be a plunger rod. The expelling sub-assembly in addition to the plunger element may comprise a drive spring or other energy store for causing the plunger element to act on the medicament container for priming and for expelling the medicament.

In addition to concerning a medicament delivery device, the present invention also concerns a medicament delivery assembly comprising the medicament delivery device of the kind described in the present text, pre-assembled with a medicament container, for example comprising a proximally arranged septum, for example arranged on a medicament container bottleneck, for example held by a crimp.

### BRIEF DESCRIPTION OF THE DRAWINGS

Embodiments of the present disclosure will now be described by way of example only and with reference to the following accompanying drawings.

The drawings show:

FIG. 1 a view of a medicament delivery assembly in an initial state;

FIG. 2 the medicament delivery assembly of FIG. 1, without the housing sleeve;

FIG. 3 the medicament delivery assembly of FIG. 1 in a longitudinal section;

FIG. 4 the medicament delivery assembly of FIG. 1 in a longitudinal section orthogonal to the section of FIG. 3;

FIG. 5 Elements of the medicament delivery assembly of FIGS. 1-4 in an exploded view;

FIG. 6 Further elements of the medicament delivery assembly of FIGS. 1-4 in an exploded view;

FIG. 7 a view of the needle guard link of the medicament delivery assembly, together with the activator;

FIG. 8 the medicament delivery assembly in seven different states during preparation;

FIG. 9 a view of the activator;

FIG. 10 a view of the needle guard link; and

FIG. 11 a view of the plunger locking member.

### DETAILED DESCRIPTION

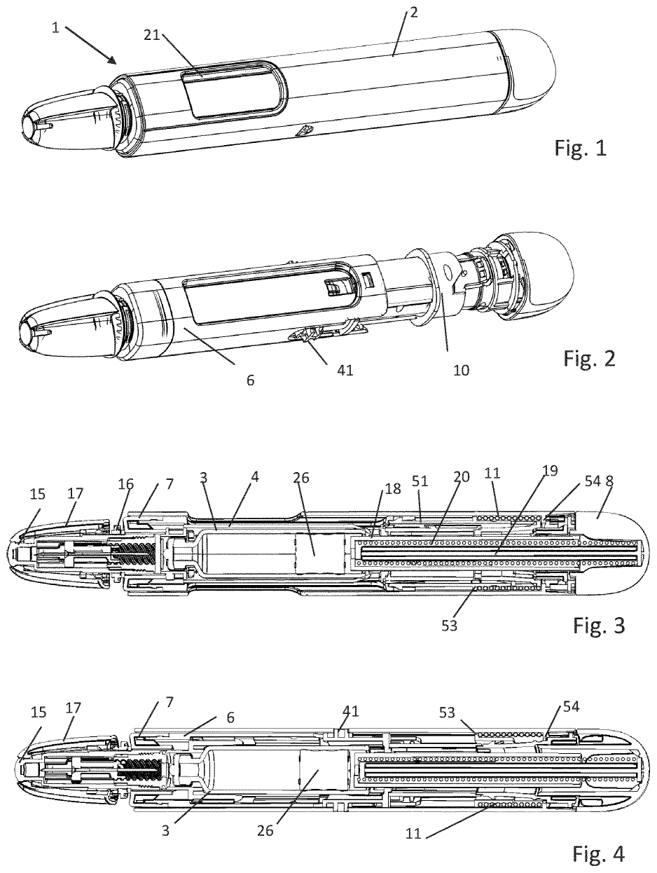

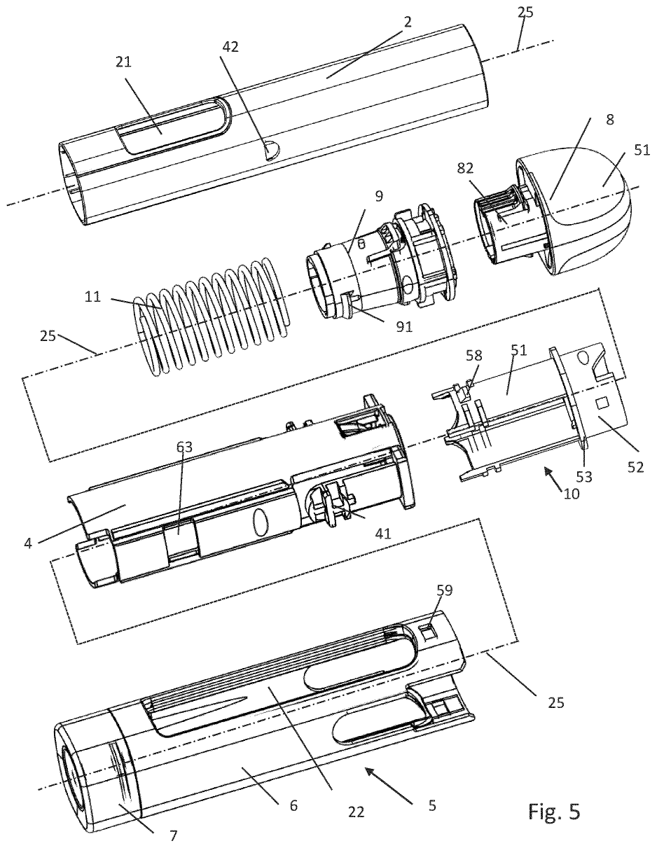

FIGS. 1-11 show a medicament delivery device of the invention. The embodiment of a medicament delivery assembly 1 shown in FIGS. 1-6 comprises the medicament delivery device of the invention and a medicament container 3. The medicament delivery device comprises a housing that has a housing sleeve 2. Within the housing sleeve 2, an optional container housing 4 for the medicament container 3 is arranged. The container housing 4 has radial protrusions 41 that fit into recesses 42 of the housing sleeve to fix the container housing 4 relative to the housing sleeve 2.

The medicament delivery device further comprises a needle guard 5 with a needle guard body 6 and a needle guard front 7. The needle guard body 6 partially extends around the container housing 4 but is movable, subject to restrictions discussed hereinafter, in axial directions relative to the housing sleeve 2 and the container housing 4. In this example, the medicament delivery device comprises an activation sub-assembly comprising the needle guard 5. In this example, an axial application movement is initiated by a distal axial movement of the needle guard 5 when a user presses the needle guard 5 in the distal direction. In one example, the axial application movement is the distal axial movement of the needle guard. Alternatively, the axial application movement is a distal axial movement of another component that is moved in the distal direction by the distal movement of the needle guard. The invention will be explained in detail below with this example, namely that the axial application movement is initiated by a distal axial movement of the needle guard.

A distal end cap 8 of the housing has a cap portion 81 extending distally from the housing sleeve 2. A proximal guiding portion 82 of the distal end cap 8 guides an activator 9 by engaging into its interior, in a manner that the axial position of the activator 9 is fixed, but that the activator 9 can be rotated around the axis 25 by a certain angle.

The medicament delivery device further comprises a needle guard link 10 that is fixedly connected to the needle guard 5 by fixing protrusions 58 engaging into according fixing cut-outs 59 of the needle guard body 6. The needle guard link 10 has two link arms 51 that extend towards proximally from a needle guard link body portion 52 and that comprise the fixing protrusions 58. The needle guard link 10 further has an annular link ledge 53 that extends outwardly from the needle guard link body portion 52. The link arms 51 accommodate a plunger locking member 12 that is arranged proximally of the activator 9 and that is rotatable about the axis 25 relative to the housing sleeve 2, the container housing 4 and the needle guard body 6. The plunger locking member 12 proximally abuts against a distal end face of the container housing 4.

Between the link ledge 53 and an inwardly extending housing ledge 54, a tensioned needle guard spring 11 is arranged.

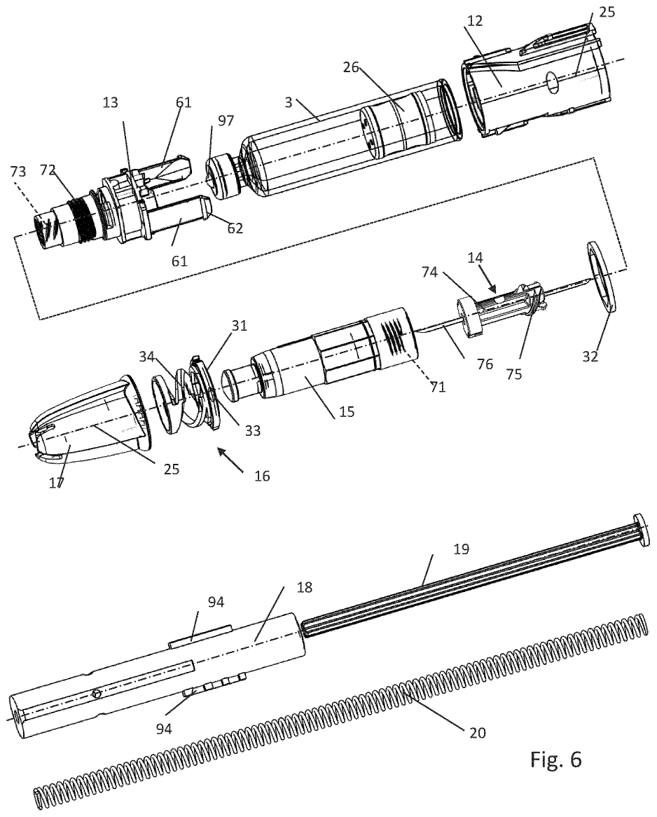

The container housing 4, when the medicament delivery assembly is assembled, houses the medicament container 3, which is depicted transparent in FIG. 6. The medicament container 3 may also in reality be transparent, and so may be the container housing 4, whereby the medicament in the medicament container 3 can be visibly inspected through a housing sleeve window 21 and a needle guard window 22.

Proximally of the medicament container 3, a retainer 13 is arranged. The retainer 13 is connected to the container housing 4 but is slidable in axial directions relative to it. More in particular, the retainer has distally extending retainer arms 61 with outward protrusions 62 at their free ends. The outward protrusions 62 engage into rectangular cut-outs 63 of the container housing 4. The rectangular cut-outs 63 have an axial extension that is larger than the axial extension of the outward protrusions 62, whereby the retainer is allowed to slide relative to the container housing 4 in axial direction by a distance that corresponds to the difference between the axial extension of the cut-outs 64 and the axial extension of the outward protrusions 62.

The medicament delivery device further comprises a needle assembly 14 housed in a needle cap 15—which needle cap 15 extends to also house a proximal portion of the retainer 13. The needle cap 15 has a cap inner thread 71 (shown only schematically in FIG. 6) that engages with a retainer outer thread 72 arranged on the proximal portion of the retainer, which proximal portion reaches into the needle cap 15 from distally. Thus, the needle cap 15 is screwed on the retainer 13. The retainer further has a retainer inner thread 73 (again shown schematically in FIG. 6). The needle assembly 14 has a needle hub 74 to which a needle 76 is fixedly connected. The needle hub 74 has a hub outer thread 75 engaging with the retainer inner thread 73. The needle hub is rotationally fixed relative to the needle cap 15 but is axially movable relative thereto.

The medicament delivery device further has an a 17 guided by a structure of the needle cap 15, with a proximal inner structure cooperating with a small proximal outward bulge of the needle cap 15 so secure the outer cap against escaping towards proximally (see for example FIGS. 3 and 4). Between the outer cap 17 and a distal portion of the needle cap 15, the medicament delivery assembly comprises a cap clutch 16. The cap clutch has a rotation coupling structure to be rotationally coupled to the needle cap 15. More in particular, the needle cap has a section with a non-round cross section on which the cap clutch 16 sits. In the depicted embodiment, this section and the rotation coupling structure have an octagonal cross section. Other cross sections, such as hexagonal, rectangular, round with axial ridges or notches, etc. would be possible, too. The cap clutch moreover may be brought into a state in which it is also rotationally coupled to the outer cap, as described hereinafter.

The needle assembly 14, the needle cap 15, the outer cap 17 and the cap clutch 16 may together with the retainer 13, the support ring 32, and, as the case may be together with sealing means and fastening clips or the like (not shown in FIG. 6) form a pre-assembled cap assembly that, when the medicament delivery assembly 1 is put together, may be secured to the other parts of the medicament delivery device after the medicament container 3 has been placed in the container housing 4 from proximally.

The medicament delivery device further comprises a plunger rod 18 extending through an interior of the activator 9 and of the needle guard link 10 and, depending on the medicament container's filling extending somewhat into the medicament container 3. In an initial configuration, before priming, the plunger rod 18 is, however, not in physical contact with the medicament container's stopper 26. The plunger rod 18 comprises an interiorly arranged drive spring 20 that in turn is guided by a spring guide 19 extending from an interior of the distal end cap 8 towards proximally.

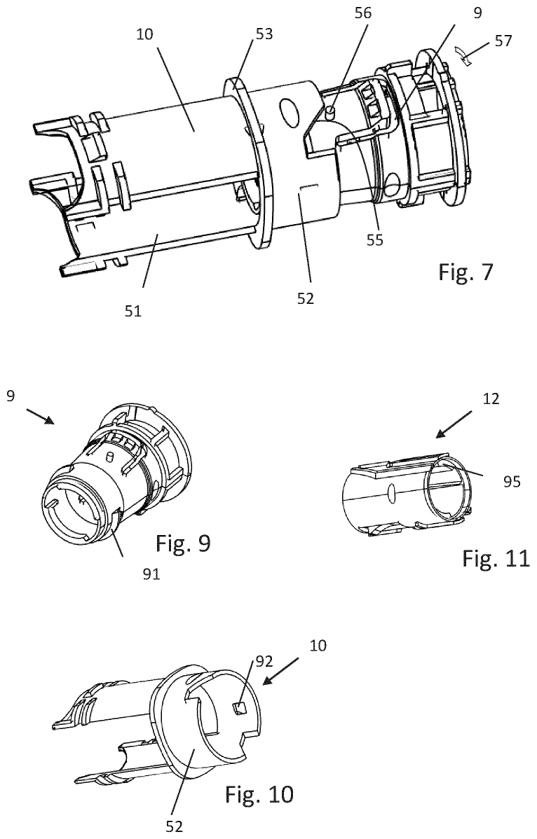

FIG. 7 shows the needle guard link 10 together with the activator 9. The needle guard link body portion 52 has a bevel 55, i.e., a section where the end face is slanted so that a ramp sloping, along the circumferential direction, axially is formed. The activator 9 has a transmission protrusion 56 that cooperates with the bevel 55 when the needle guard link 10 is pushed into an axial direction towards distally. In particular, when the needle guard link is moved towards distally, the bevel 55 pushes against the transmission protrusion 56, and thereby, because the activator 9 cannot move towards distally, causes a torque and thus a rotational movement of the activator 9 in the direction indicated by arrow 57.

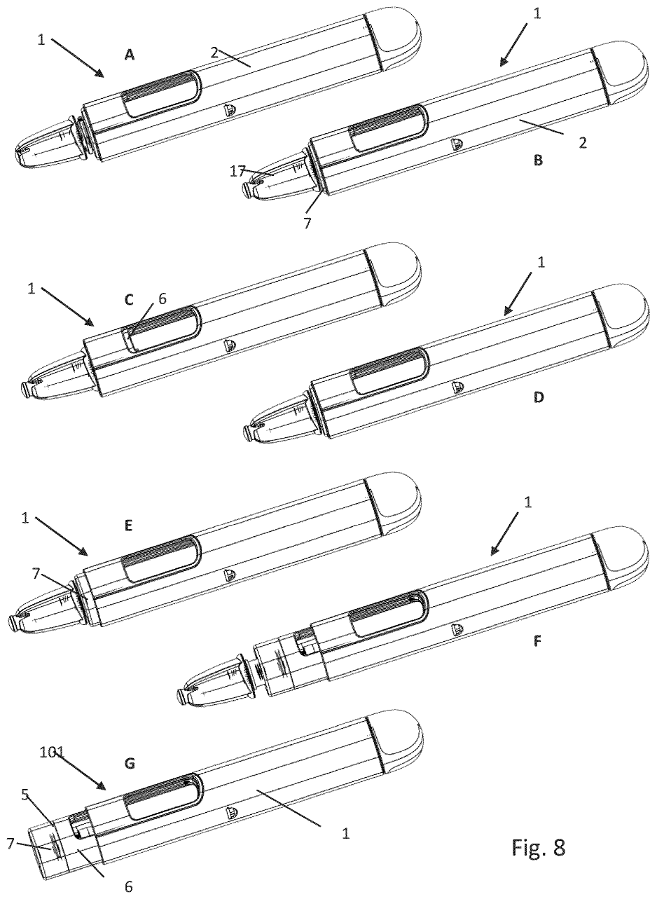

FIGS. 1-4 show an initial configuration in which the medicament delivery assembly is delivered to the user. In this configuration, the medicament delivery device is provided with a medicament container 3 in the container housing 4, and with the plunger rod 18 in the distal-most position relative to the container housing 4, with the drive spring 20 tensioned. The needle guard 5 is in the position shown in FIGS. 1-4, with also the needle guard spring 11 pre-tensioned. FIG. 8 depicts, in panels A through G, a sequence for preparation of the medicament delivery assembly 1 for use.

For activating, the user pushes the outer cap 17—and thereby also the needle cap 15—from the initial configuration (panel A) towards distally until a distal end face of the outer cap abuts against the coupling portion 31 of the cap clutch 16 (panel B). Then, she pushes the outer cap 17 further to displace, via the coupling portion 31 of the cap clutch, both, the activation sub-assembly that comprises the needle guard 5 together with the needle guard link 10 and, by means of a support ring 32 engaging the needle cap 15, the cap assembly, comprising the needle cap 15, the needle sub-assembly 14 and the retainer 13, with the medicament container 3 (panel C). This movement towards distally is confined by the interplay between the outward protrusions 62 of the retainer 13 and the cut-outs 63 in the immobile container housing 4. When the proximal end position defined by the cut-outs 63 is reached, the medicament container 3 has been moved to a position in which the stopper 26 is in physical contact—or almost in physical contact—with the proximal end of the plunger rod 18.

The movement of the activation sub-assembly comprising the needle guard 5 with the needle guard link 10 is called ‘application movement’ in the present text. It will further cause, by the mechanism explained referring to FIG. 7, a rotation of the activator 9 about the axis 25 by an angle of for example less than 90°.

This rotation will firstly cause an outward ledge 91 of the activator 9 (FIG. 9) to cease to be a stop for an axial movement of the needle guard link 10 (and thereby of the needle guard 5 fixedly connected thereto). This is because the rotation causes the outward ledge 91 to be out of alignment with an inward protrusion 92 of the needle guard link 10 (FIG. 10). As a result, the needle guard link 10 may, together with the needle guard 5 travel, driven by the needle guard spring 11, towards proximally until the needle guard front 7 is stopped (for example by the support ring 32 engaged with the needle cap 15) as soon as the user allows so (panels D and E of FIG. 8).

Secondly, this rotation causes a control protrusion 94 of the plunger rod 18 to get out of engagement with an inward ledge 93 of the activator 9. Thereby, the inward ledge 93 serves as the control structure. Because the control protrusion gets out of engagement with the inward ledge, the plunger rod 18, driven by the drive spring 20, moves slightly towards proximally and thereby is allowed to exert a force on the stopper 26. Since the medicament container 3 is closed, this will not cause any dispensing of any medicament but will cause the content of the medicament container to be under a pressure.

Thereafter, the user may remove the outer cap 17 together with the needle cap 15 and thereby at the same time cause the needle 76 to penetrate a proximally arranged septum 97 of the medicament container 3. The according mechanism is for example described in WO 2013/089616: The coupling portion 31 of the cap clutch 16 has an engagement structure 33. Because the needle guard presses the coupling portion 31, against a bias force by a clutch biasing means 34, into the outer cap 17, and/or because the user presses the outer cap towards distally far enough, the engagement structure engages with an inner coupling structure of the outer cap and thereby rotationally couples the outer cap 17 with the cap clutch 16, and consequently also with the needle cap 15 that is rotationally coupled with the cap clutch 16 by having the with the non-round cross section on which the cap clutch 16 sits.

Thus, a rotation of the outer cap 17 in this state (panels E and F of FIG. 8) causes the inner and outer caps to be unscrewed. At the same time, the rotation of the needle cap 15 causes the needle sub-assembly 14, which is rotationally locked to the needle cap 15 while the retainer 13 having the retainer inner thread 73 does not rotate, to be advanced towards distally for the needle 76 to pierce the septum.

This will allow the pressure inside the medicament container 3 to be released for finishing the priming process. The plunger rod 18 slightly advances towards proximally, pushing the stopper 26 until the control protrusion 94 is stopped by a control abutment 95 of the plunger locking member 12 (FIG. 11).

Removal of the outer cap 17 with the needle cap 15 will allow the needle guard 5, driven by the needle guard spring 11, to move to the fully extended position (panel G of FIG. 8) in which the needle guard protects the needle 76, from which the needle cap 15 has been removed. The resulting remaining needle guard assembly 101 (without the outer cap 17, the needle cap 15 and the cap clutch 16) is then ready for use.

For use, the user presses the remaining needle guard assembly 101 against a dose delivery site to displace the needle guard 5 towards distally, whereby a rotation of the plunger locking member 12 will be caused. This in turn causes the plunger rod 18 to be released and to be capable of being driven towards proximally, for the stopper 26 to expel the medicament from the medicament container through the needle 76 that has penetrated the user's tissue.

Various modifications are possible:

For example, the drive spring could be replaced by a pneumatic power pack, for example as disclosed in WO 2020/0056068.

Also, the mechanism by which the outer cap and the needle cap are coupled to teach other, can be different than disclosed. It would even be possible to modify the medicament delivery assembly in a manner that no separate outer cap is required any more, for example if a security mechanism impedes a twisting off of the (inner) cap prior to activation.

Even more, it is not excluded that the functions of the activator and of the plunger locking member are combined in one single part.

Further, it is not required that the functions of the activation sub-assembly and of the shielding the needle after removal of the cap are taken over by the same part (the needle guard). Rather, the activation sub-assembly may comprise parts separate from the needle guard—for example the mentioned button at the distal end of the housing—or alternatively another structure, separate from the needle guard—that is to be pushed towards proximally, for example by pushing an outer cap.

As a further modification, the housing may have a structure that directly accommodates the medicament container, without there being a dedicated container housing.

An even further modification concerns the needle guard. Instead of the medicament delivery device having an initially separate needle guard link 10 coupled to the needle guard, the structure of the needle guard link may be integrated in the needle guard. For example, the needle guard may have a proximodistal extension corresponding to the extension of the needle guard assembled with the needle guard link, and the needle guard may comprise, at a distal end thereof, the bevel 55.

In addition, or as an alternative, also the needle guard front may be one-piece with the needle guard body.

The drug delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-16. (canceled)

17. A medicament delivery device, comprising a housing having a proximal end and a distal end and defining an axis extending proximodistally, and an expelling unit for expelling a medicament from a medicament container seated within the housing, the expelling unit comprising plunger element equipped to be driven in an axial direction while acting on the medicament container to expel the medicament, the medicament delivery device further comprising an activator, wherein rotation of the activator about the axis by an angle is a pre-condition for the expelling unit to expel the medicament, and wherein the medicament delivery device comprises an activation sub-assembly, the activation sub-assembly being configured to be caused, by a user, to be subject to an axial application movement, wherein the activation sub-assembly cooperates with the activator to rotate the activator about the axis when being subject to the axial application movement.

18. A medicament delivery device according to claim 17, further comprising an outer cap removeable by the user together with a needle cap, wherein the activation sub-assembly is configured to be caused, by the user, to be subject to the axial application movement by the user pushing the needle cap distally.

19. A medicament delivery device according to claim 17, wherein the activation sub-assembly comprises a needle guard and a needle guard link, and wherein when the needle guard link is moved towards distally by the axial application movement, the needle guard link pushes against the activator, and thereby causes the rotational movement of the activator.

20. The medicament delivery device according to claim 17, wherein one of the activator and the activation sub-assembly comprises a bevel and the other one comprises a bevel engaging structure facing towards the bevel, wherein an axial position of the activator is fixed relative to the housing, so that a pressing force causing the axial application movement causes the bevel and the bevel engaging structure to be pressed against each other, whereby a torque on the activator relative to the activation sub-assembly is generated, the torque causing the rotation of the activator.

21. The medicament delivery device according to claim 20, wherein the bevel comprises a ramp running axially and circumferentially.

22. The medicament delivery device according to claim 20, wherein the bevel engaging structure comprises a transmission protrusion being a radial protrusion.

23. The medicament delivery device according to claim 17, further comprising a needle, a needle cap protecting the needle and an outer cap extending around at least a portion of the needle cap, wherein medicament delivery requires removal of the needle cap together with the outer cap by a rotation of the needle cap, and wherein the medicament delivery device is configured such that a rotational coupling between the needle cap and the outer cap is only possible upon completion of the axial activation movement.

24. The medicament delivery device according to claim 23, wherein the rotation of the needle cap also causes the needle to be moved towards distally so as to pierce a septum of the medicament container.

25. The medicament delivery device according to claim 17, wherein the activator comprises a control structure, wherein the rotation of the activator causes the control structure to get out of engagement with a control protrusion of the plunger element, whereby the rotation releases the plunger element from being locked in a distal position and thereby allows the plunger element to be moved towards proximally by a drive force.

26. The medicament delivery device according to claim 25, further comprising a control abutment, the control abutment limiting a movement of the plunger element towards proximally after the plunger element has been released from being locked in the distal position.

27. The medicament delivery device according to claim 26, comprising a rotatable plunger locking member separate from the activator, wherein the plunger locking member comprises the control abutment.

28. The medicament delivery device according to claim 17, comprising a/the needle protected by a needle cap and comprising a needle guard configured to be moved to an extended position in which the needle guard protects the needle after removal of the needle cap.

29. The medicament delivery device according to claim 28, wherein the activation sub-assembly comprises the needle guard.

30. The medicament delivery device according to claim 17, wherein the medicament container is movable relative to the housing by the application movement, together with the activation sub-assembly, for bringing the plunger element into contact or closer to a stopper of the medicament container.

31. The medicament delivery device according to claim 30, wherein the housing comprises a container housing for accommodating the medicament container, and wherein the medicament container is movable relative to the container housing by the application movement.

32. A medicament delivery assembly, comprising the medicament delivery device according to claim 17, and further comprising the medicament container assembled with the medicament delivery device.
